(19) United States
(12) Patent Application Publication
(10) Pub. No.: US 2020/0275988 A1
(43) Pub. Date: Sep. 3, 2020

(54) IMAGE TO WORLD REGISTRATION FOR MEDICAL AUGMENTED REALITY APPLICATIONS USING A WORLD SPATIAL MAP

(71) Applicant: THE JOHNS HOPKINS UNIVERSITY, Baltimore, MD (US)

(72) Inventors: Alex A. Johnson, Baltimore, MD (US); Kevin Yu, Baltimore, MD (US); Sebastian Andress, Baltimore, MD (US); Mohammadjavad Fotouhighazvin, Baltimore, MD (US); Greg M. Osgood, Baltimore, MD (US); Nassir Navab, Baltimore, MD (US); Mathias Unberath, Baltimore, MD (US)

(21) Appl. No.: 16/753,076

(22) PCT Filed: Oct. 2, 2018
(86) PCT No.: PCT/US2018/053934

§ 371 Date: Apr. 2, 2020

Related U.S. Application Data
(60) Provisional application No. 62/566,771, filed on Oct. 2, 2017.

(51) Int. Cl. A61B 90/00; G02B 27/01; G16H 30/40

(52) U.S. Cl. CPC A61B 90/39; G16H 30/40; G02B 27/017

(57) ABSTRACT

The present invention is directed to a system and method for image to world registration for medical reality applications, using a world spatial map. This invention is a system and method to link any point in a fluoroscopic image to its corresponding position in the visual world using spatial mapping with a head mounted display (HMD) (world tracking). On a projectional fluoroscopic 2D image, any point on the image can be thought of as representing a line that is perpendicular to the plane of the image that intersects that point. The point itself could lie at any position in space along this line, located between the X-Ray source and the detector. With the aid of the HMD, a virtual line is displayed in the visual field of the user.

### CROSS REFERENCE TO RELATED APPLICATIONS

This application claims the benefit of U.S. Provisional Patent Application Ser. No. 62/566,771 filed Oct. 2, 2017, which is incorporated by reference herein, in its entirety.

### FIELD OF THE INVENTION

The present invention relates generally to imaging. More particularly, the present invention relates to an image registration system for medical augmented reality applications using a world spatial map.

### BACKGROUND OF THE INVENTION

Image guided procedures are prevalent throughout many fields of medicine. Certain procedures using image guidance are performed without registration of the images to a reference. For example, use of fluoroscopy during many orthopedic procedures is done in this manner. This means that the C-arm acquires a fluoroscopic image of a specific part of the patient and the surgeon simply looks at this fluoroscopic image on a monitor and uses this information to guide completion of the procedure. Unregistered image guided procedures are common using a variety of imaging modalities including ultrasound, fluoroscopy, plain radiography, computed tomography (CT), and magnetic resonance imaging (MRI). However, sometimes surgeons need more detailed information and more specific guidance when performing procedures. This can be achieved via use of registration of the imaging to references such as fiducials fixed to the patient or attached to tools. There are many different types of image registration including optical and electromagnetic methods. These registration methods can be very helpful and are used regularly throughout medicine; however, there are certain drawbacks associated with each, such as intereference of the line of sight required for optical registration, invasive nature of placing fiducials, change to traditional workflow, and expense. Most of these registration methods are “outside in” meaning that the imaging and trackers are focused towards the patient and the fiducials localized within the surgical field. Rather than continuing with this traditional “outside-in” registration, this invention presents a novel “inside-out” registration method.

With traditional unregistered C-arm fluoroscopy, only a single plane can be viewed at a time (anterior to posterior, lateral, etc.) by the surgeon, and these images give little information about the depth of the structures or location of structures inside the image in relation to the world (the visual scene of the surgeon). Simple methods can be used by the surgeon to help link (in the mind of the surgeon) the position of structures within the fluoroscopic image to the world. One simple way of doing this is to hold a radio-opaque tool (such as a wire) in the X-Ray beam. A radiographic shadow of the tool is then seen in the image and the surgeon can also visually see the tool in the world, thus understanding the position of the tool in both the fluoroscopic space and the world space. However, this only gives positional information in the current plane of the fluoroscopic image, giving no depth information about the position of the tool. This process can be repeated in another fluoroscopic plane (usually orthogonal to the first). Then, the position of the tool is known in both planes, and the surgeon can imagine (again in his or her mind) where the anatomical structure of interest is located inside the body. Because the tool must be moved to be visible when the new fluoroscopic plane, surgeons will sometimes draw a line or cross on the surface of the patient's body (anterior for example) to keep a physical “record” of a where the tool was positioned in that fluoroscopic plane. This can be repeated for the second position of the tool on a different surface of the patient's body (lateral for example). If one cross is drawn on the anterior surface of the body, and another on the lateral surface, then the surgeon can imagine lines extending perpendicular to the plane of each cross that intersect at the point of interest inside the patient's body. This process is problematic for the following two reasons: 1) It requires the surgeon to mentally visualize or imagine the point at which the two imaginary lines intersect within the body. This task is difficult, inaccurate, and imprecise. 2) It requires that a known portion of the radio-opaque tool be at the exact desired position of interest in the fluoroscopic image on both planes. This can require the surgeon to take multiple images in a single plane to get the tool positioned at the correct point on the fluoroscopic image. This can be time consuming and generates additional radiation. However, this traditional method highlights how surgeons currently attempt to link the position of anatomy visualized in the imaging to their position in the environment.

Accordingly, there is a need in the art for a system and method to link the image frame of reference (what the surgeon sees in the fluoroscopic images) to the world frame of reference (what the surgeon sees in the surgical field). This may be accomplished by image to world registration for medical reality applications using a world spatial map as is described herein.

### SUMMARY OF THE INVENTION

The foregoing needs are met, to a great extent, by the present invention which provides a system for image to world registration including a world spatial map. The proposed mode of viewing and interacting with registered information is via optical see through head mounted display (HMD). The system also includes a non-transitory computer readable medium programmed for receiving image information. The system is also programmed for linking any point in the image information to a corresponding point in a visual field displayed by the head mounted display and displaying a visual representation of the linking of the image information to the corresponding point in the visual field displayed by the head mounted display. The system allows for interacting with the two-dimensional image space and viewing this interaction in the three-dimensional world space. Thus as points and lines are templated on the radiographic image, these are visualized in the world space as three-dimensional lines and planes. Such registration and visualization may be used for targeting, navigation, templating, and measuring in surgery as well as “imaging mapping” or “stitching” of medical images. This method can be applied to 2D planar imaging (fluoroscopic) or 3D axial imaging (CT, MRI) in combination with traditional 2D-3D registration methods.

In accordance with an aspect of the present invention, the system includes generating visual representations in multiple planes corresponding to multiple planes in the image information. The virtual representation can take a variety of forms, including but not limited to a point, a line, a column, or a plane. The system can also include a radiovisible augmented reality (AR) marker.

In accordance with another aspect of the present invention, a device for labeling in an image includes an augmented reality marker configured to be recognizable by a camera such that virtual information can be mapped to the augmented reality marker. The device also includes a radiographic projection configured to be visually detectable in a fluoroscopic image. The augmented reality marker has an orientation in a world frame of reference such that it is linked to an orientation in an image frame of reference. The augmented reality marker includes a projectional appearance showing how an X-Ray beam impacted it in a physical world. The radiographic projection is recognizable by a camera such that virtual information can be mapped to the radiographic projection. The virtual information is configured to be displayed via a head mounted display (HMD). An orientation of the augmented reality marker in a world frame of reference is linked to its orientation in an image frame of reference. An orientation and relation of an X-ray beam to the augmented reality marker is determined from a projectional appearance of the augmented reality marker in a resultant radiographic image. The augmented reality marker is configured to be positioned such that translations are performed in a same plane as a projectional image.

The present invention is directed to an easy-to-use guidance system with the specific aim of eliminating any potential roadblocks to its use regarding system setup, change in workflow, or cost. The system conveniently applies to image guided surgeries, and provides support for surgeon's actions through an augmented reality (AR) environment based on optical see-through head-mounted displays (HMD) that is calibrated to an imaging system. It allows visualizing the path to anatomical landmarks annotated on 2D images in 3D directly on the patient. Calibration of intra-operative imaging to the AR environment may be achieved on-the-fly using a mixed-modality fiducial that is imaged simultaneously by a HMD and a C-arm scanner; however, calibration and registration is not limited to this method with the use of the mixed-modality fiducial. Therefore, the proposed system effectively avoids the use of dedicated but impractical optical or electromagnetic tracking solutions with 2D/3D registration, complicated setup or use of which is associated with the most substantial disruptions to the surgical workflow.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIGS. 1A and 1B illustrate exemplary positions of the C-arm, according to an embodiment of the present invention.

FIG. 2A illustrates an image view of an AP fluoroscopic image of the left hip including a radiographic fiducial. The location of the fiducial is indicated by the arrow.

FIG. 2B illustrates an augmented image view of an anatomical femur model showing a virtual line extending through the location of the visible marker.

FIG. 3 illustrates an AP fluoroscopic image of the left hip including radiographic fiducial. The desired target position is indicated by the tip of the arrow.

FIG. 4 illustrates an augmented view of an anatomical femur model showing a virtual line extending through the original location of the fiducial and the desired location at the tip of the greater trochanter.

FIG. 5A illustrates an image view of an augmented reality marker visible on fluoroscopic view.

FIG. 5B illustrates an image view of a translation of virtual line from the center of the fiducial to the desired target location.

FIG. 6A illustrates a perspective view of a virtual line in the world frame of reference intersecting the desired point on the fluoroscopic image frame of reference.

FIG. 6B illustrates an augmented view showing virtual line intersecting the AP plane in a mock OR scenario.

FIG. 7A illustrates an image view of a lateral fluoroscopic image of the left hip including radiographic fiducial indicated by blue arrow.

FIG. 7B illustrates an augmented view of an anatomical femur model showing a virtual line parallel to the floor extending through the location of the visible marker corresponding to the radiographic fiducial.

FIG. 8A illustrates a lateral fluoroscopic image of the left hip including radiographic fiducial. Desired position indicated by tip of blue arrow.

FIG. 8B illustrates an augmented view of an anatomical femur model showing a virtual line extending through the original location of the fiducial

FIG. 9 illustrates a schematic diagram of virtual lines in the world frame of reference intersecting the desired points on the AP and lateral fluoroscopic images.

FIG. 10 illustrates an augmented view showing lines intersecting at the target point within the body.

FIG. 11 illustrates a schematic diagram of spatial transformations for on-the-fly AR solutions.

FIGS. 12A-12D illustrate image views of steps in the creation of the multi-modality marker.

FIGS. 13A and 13B illustrate image views of source position of the C-Arm shown as a cylinder and virtual lines that arise from annotations in the fluoroscopy image.

FIG. 14 illustrates a schematic diagram phantoms used in studies assessing the performance of the system in a surgery-like scenario.

FIG. 15A illustrates a perspective view of an augmented reality marker, according to an embodiment of the present invention, and FIG. 15B illustrates an image view of a radiographic projection of the augmented reality marker as it would appear in a fluoroscopic image.

FIG. 16A illustrates a perspective view of an augmented reality marker recognizable to camera with virtual information mapped to it.

FIG. 16B illustrates an image view of a theoretical radiographic projection of the same augmented reality marker as it would appear in a fluoroscopic image.

FIGS. 17A-17F illustrate image and schematic views of the projectional appearance of the AR marker on the radiograph shows how the x-ray beam impacted it in the physical world frame.

FIG. 18 illustrates a perspective view of a fluoroscopic image including an X-Ray visible AR marker with virtual information mapped to the radiographic projection.

FIGS. 19A-19F illustrate image and schematic views of an embodiment of the present invention.

FIGS. 20A and 20B illustrate image views of exemplary images with which the physician can interact.

FIGS. 21A and 21B illustrate image views of exemplary images with which the physician can interact.

FIG. 22 illustrates a perspective view of a radiovisible Augmented Reality Marker.

FIGS. 23A and 23B illustrate image views of fluoroscopic images demonstrating the radiographic projection of the lead AR marker.

FIG. 24 illustrates a perspective view of a radiovisible AR marker with radiotranslucent “well” filled with liquid contrast agent.

FIG. 25 illustrates an image view of a fluoroscopic image with radiographic projection of radiovisible AR marker made from radio-translucent “well” filled with liquid contrast agent.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The present invention is directed to a system and method for image to world registration for medical augmented reality applications, using a world spatial map. This invention is a method to link any point in a image frame of reference to its corresponding position in the visual world using spatial mapping with a head mounted display (HMD) (world tracking). Flouroscopy is used as an example imaging modality throughout this application; however, this registration method may be used for a variety of imaging modalities. Rather than “scanning” or “tracking” the patient or tools within the field and linking this to the imaging, the new inside-out registration system and method of the present invention scans the environment as a whole and links the imaging to this reference frame. This has certain advantages that are explained throughout this application. One advantage of this registration method is that it allows a similar workflow to what surgeons currently use in nonregistered image guided procedures. This workflow is described in the following section.

According to another aspect of the present invention, the HMD uses Simultaneous Localization and Mapping (SLAM). The system includes creating a template on the imaging frame of reference in two dimensions and subsequently allowing the user to visualize this template in three dimensions in the world space. The system includes scanning the environment as a whole and linking the image information to this reference frame. The system also includes generating a workflow that mimics a surgeons preferred workflow. The system displays a virtual line that is perpendicular to a plane of the image that intersects a point of interest. The point of interest lies at any point along the virtual line. The system displays the virtual line in a user's field of view in a head mounted display. The includes overlapping world spatial maps and a tracker rigidly fixed to a medical imaging system.

On a projectional fluoroscopic 2D image, any point on the image can be thought of as representing a line that is perpendicular to the plane of the image that intersects that point. The point itself could lie at any position in space along this line, located between the X-Ray source and the detector. With the aid of the HMD, a virtual line is displayed in the visual field of the user.

The lines in the visual field of the user can be drawn in multiple planes corresponding to each fluoroscopic plane that is acquired. If lines are drawn through points on two orthogonal fluoroscopic images for example, then the intersection of these points (corresponding to anatomical structure of interest) can be visualized. The intersection of these lines defines a single point in space that corresponds to points chosen on the 2 orthogonal images (this assumes that the points chosen on the 2 images do in fact overlap, i.e. they are the same structure viewed from two different vantage points).

One aspect of the present invention allows the user to template on the imaging frame of reference in two dimensions, but visualize this template in three dimensions in the world space. Visualizing a single point in space (the intersection of two lines) defines a single visual target on the anatomical structure, but it does not define the geometric axes of the structure. For example, an exact point on the greater trochanter of the femur can be visualized, but the longitudinal axis of the femur is not visualized. However, if lines chosen on the fluoroscopic image two dimensional space and corresponding planes are visualized in the three dimensional world space, then anatomical axes can also be visualized. This logic can be extended to more and more complex structures so as virtual anatomic models (or actual patient preoperative 3D volumes) can be overlayed at the correct position in space based upon the fluoroscopic images. Furthermore, the method of the present invention allows for correlation of distant fluoroscopic planes through multiple acquisitions.

Some HMD's allow for an augmented view of the world where digital models and images can be overlayed over the visual view of the user. Traditionally, these virtual objects have been positioned using an augmented reality “marker”, an object that the camera of the HMD can visualize to understand where to position the virtual object in space. However, some HMD's are able to perform “markerless” augmented reality and can track the world environment and “understand” the relational position of virtual objects to real word objects and the environment as a whole without using markers. This ability is powerful because it no longer requires the augmented reality (AR) marker to be in the field of view of the HMD camera in order to perform the augmentation. Using this world tracking, virtual objects can be permanently locked in place in the environment. The stability of these locked virtual objects depends upon how well the environment was tracked among other factors.

A plethora of surgical navigation systems have been developed in order to give surgeons more anatomical and positional information than is available with the naked eye or single plane fluoroscopic imaging. Two components make up most of these navigation systems: 1) a method of registration of preoperative or intraoperative imaging to the body and 2) a method to display this information to the surgeon.

Registration is a complex problem that has been and continues to be extensively studied by the scientific community. Modem registration systems commonly rely on optical tracking with reflective marker spheres. These spheres are attached to rigid fiducials that are drilled into known locations in bone. The spheres are tracked in real time by an optical tracking system, thus allowing for a registration of preoperative imaging or planning to the current position of the body. The downside of such optical tracking is that the spheres must be visible at all times to the camera, fiducials must be rigidly fixed, and the system is very sensitive to undesired movement. Other registration systems use information from single plane fluoroscopic images to register preoperative 3D imaging, known as 2D-3D registration. This method is powerful in that it can provide 3D information with the acquisition of 2D images; however, it usually requires preoperative 3D imaging (CT, MRI) to be obtained, and the registration process can be time consuming. Furthermore, both of these methods require changes to the traditional surgical workflow, additional hardware in the operating room, or software interface with imaging machines. Recent methods have been studied that combine these two registration approaches into one automatic system for registration. Some proposed systems use spherical infrared reflectors used in conjunction with radiopaque materials that are visible on X-Ray projections, thus combining 2D-3D registration with optical registration. While introducing the concept of a mixed-modality marker, these systems still require real time tracking of optical markers. Another solution introduces a radiographic fiducial that can automatically be recognized in fluoroscopic images to determine the pose of the C-arm in 3D space. While helpful in determining how the X-Ray beam impacts the fiducial, this method does not link the fiducial to the world frame of reference. The system and method of the present invention is different from prior registration methods in the following ways.

The system and method of the present invention utilizes spatial mapping of the environment. Rather than linking the fluoroscopic image to the frame of the optical tracking system, the proposed system and method links the fluoroscopic image to the actual environment through the spatial mapping of the room. This may be accomplished in variety of ways including but not limited to using a mixed modality marker (X-Ray/AR rather than X-Ray/infrared), point localization with hand gestures, head movement, or eye tracking (using the hand or a head/eye driven virtual cursor to define an identifiable position in the visible field that is also recognizable on the fluoroscopic image, i.e. the tip of a tool), as well as the linking of multiple spatial mapping systems. For example, a spatial mapping tool may be rigidly mounted on an imaging system (a C-arm for example). The spatial mapping acquired by this tool mounted to the imaging system can be linked to the spatial mapping acquired from the HMD worn by the user. Thus all images acquired by the imaging system may be registered to the same spatial map that the user may visualize. The system and method of the present invention allows every image taken by the medical imaging system to be registered to actual positions locked to the spatial mapping of the room.

One method of performing this image to world registration via world spatial map is using a mixed modality fiducial that may be visualized in the image frame of reference and the world frame of reference at the same time. This method is described in detail herein; however, this is a single method of image to world registration using a world spatial map and does not attempt to limit the image to world registration via spatial mapping to this specific method.

Mixed Modality Method:

The following method describes how an image can be registered to a world spatial map using only an HMD (with spatial mapping), a standard fluoroscopy machine, and a single mixed modality fiducial. An orthopaedic example of finding the starting point for intramedullary nailing of the femur is included for simplicity to describe the method. However, this example is not meant to be considered limiting and is only included to further illustrate the system and method of the invention.

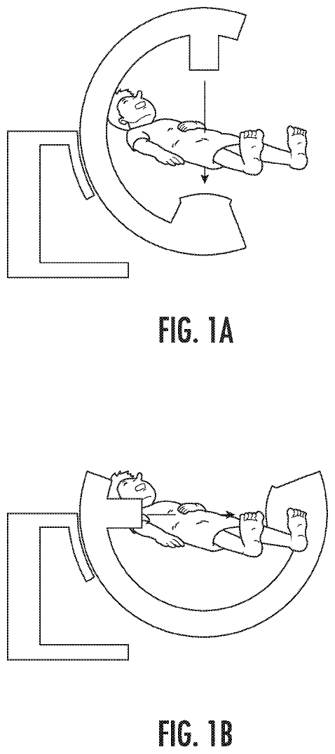

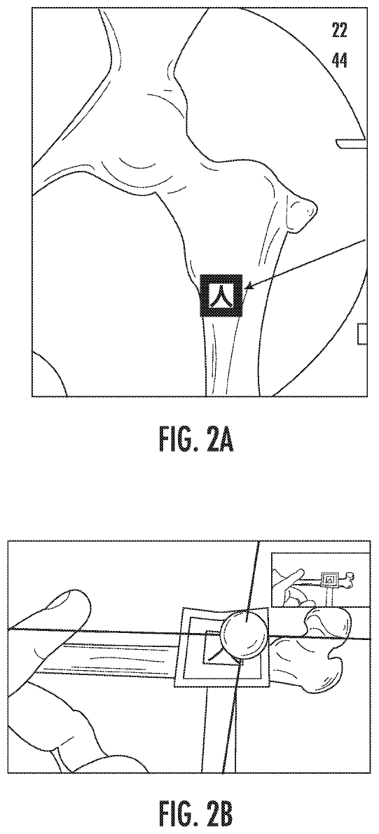

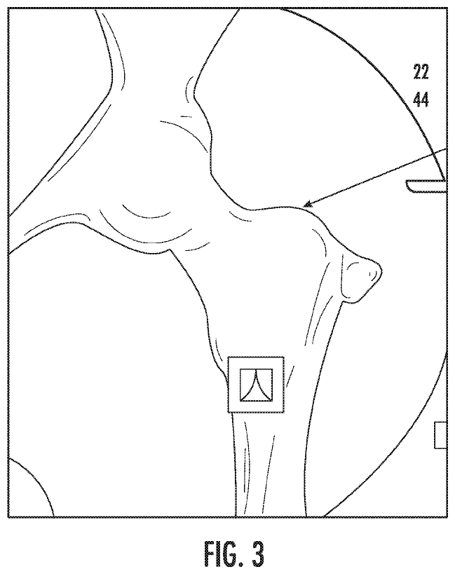

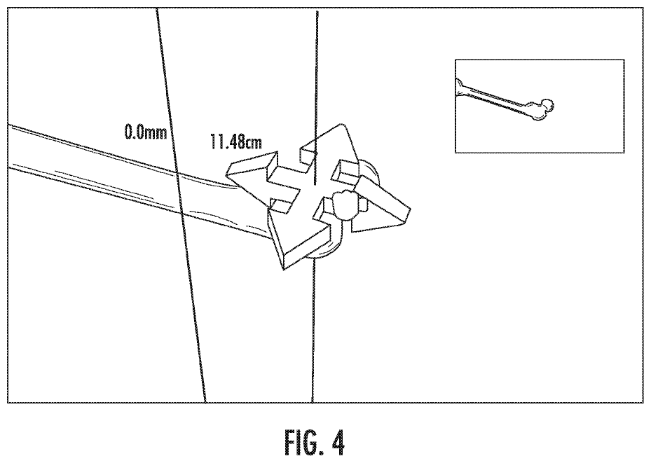

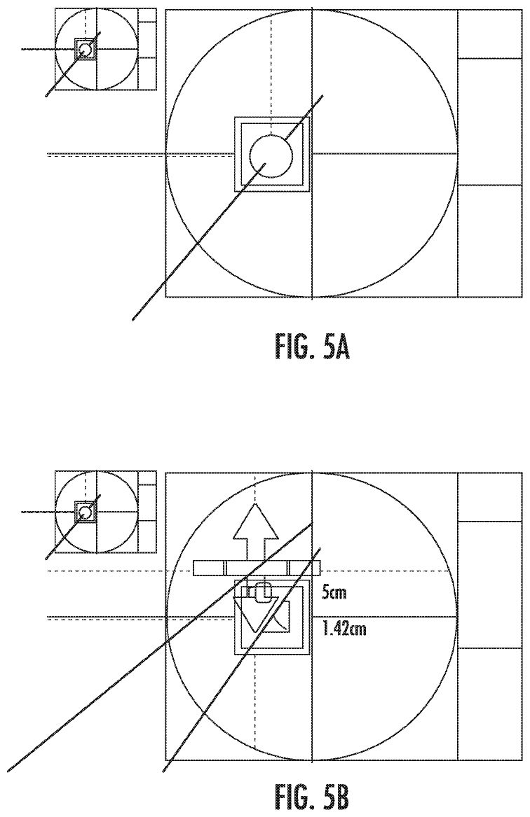

FIGS. 1A and 1B illustrate exemplary positions of the C-arm, according to an embodiment of the present invention. In an exemplary embodiment, the C-arm is positioned perpendicular to the table and floor of the room for the AP image and parallel to the table and floor of the room for the lateral image, as illustrated in FIGS. 1A and 1B. The procedure begins with acquisition of an anterior to posterior (AP) image with the radiographic fiducial/AR marker in the X-Ray beam. In such a case, the HMD recognizes the position of the radiographic fiducial/AR marker and “world locks” it to the spatial map. It then draws a virtual line perpendicular to the floor that intersects this point. At this time in the procedure, the user would see a fluoroscopic image with the fiducial readily detectable as illustrated in FIG. 2A and a virtual line in the environment extending from source to detector and through the patient as illustrated in FIG. 2B. FIG. 2A illustrates an image view of an AP fluoroscopic image of the left hip including a radiographic fiducial. The location of the fiducial is indicated by the arrow. FIG. 2B illustrates an augmented image view of an anatomical femur model showing a virtual line extending through the location of the visible marker. This line intersects the point on the fluoroscopic image at which the fiducial is located. However, this may or may not be the desired starting point or target for the surgery. It is then necessary for the surgeon to locate the desired point on the AP fluoroscopic image. For a trochanteric entry nail, this is at the medial tip of the greater trochanter as seen in FIG. 3. FIG. 3 illustrates an AP fluoroscopic image of the left hip including radiographic fiducial. The desired target position is indicated by the tip of the arrow. Then, in the plane of the fluoroscopic image, the position of the fiducial is translated to the desired position of the target as in FIG. 4. FIG. 4 illustrates an augmented view of an anatomical femur model showing a virtual line extending through the original location of the fiducial and the desired location at the tip of the greater trochanter. FIG. 5A illustrates an image view of an augmented reality marker visible on fluoroscopic view. FIG. 5B illustrates an image view of a translation of virtual line from the center of the fiducial to the desired target location.

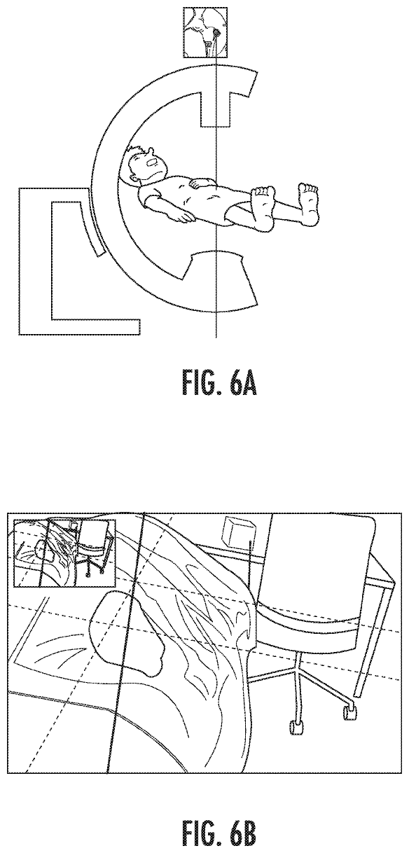

Once the translation has occurred, the user will then be viewing a virtual line that intersects the exact point on the patient. This virtual line corresponds to the desired target point on the fluoroscopic image, as illustrated in FIGS. 6A and 6B: FIG. 6A illustrates a perspective view of a virtual line in the world frame of reference intersecting the desired point on the fluoroscopic image frame of reference. FIG. 6B illustrates an augmented view showing virtual line intersecting the AP plane in a mock OR scenario.

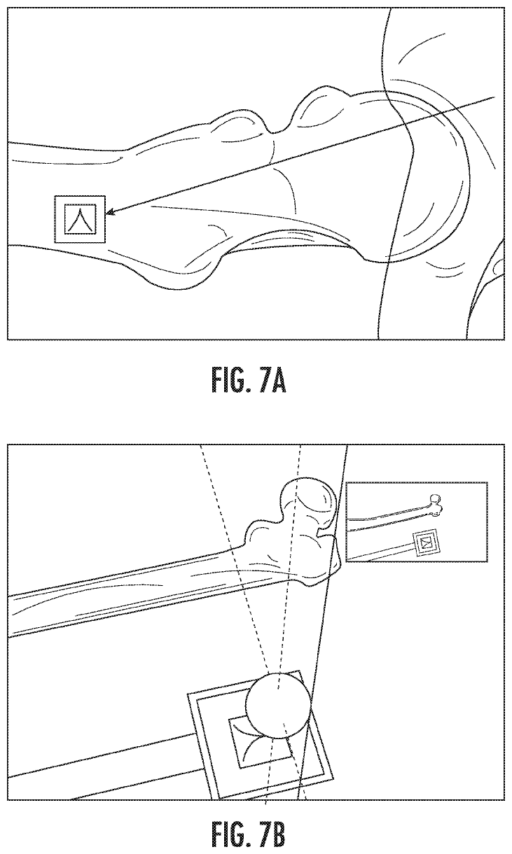

This entire process can then be repeated for any additional fluoroscopic plane. For this example, the C-arm is then positioned in the lateral position with the beam parallel to the floor of the room. At this point, a lateral fluoroscopic image of the hip is obtained with the fiducial included in the X-Ray beam as seen in FIGS. 7A and 7B. FIG. 7A illustrates an image view of a lateral fluoroscopic image of the left hip including radiographic fiducial indicated by blue arrow. FIG. 7B illustrates an augmented view of an anatomical femur model showing a virtual line parallel to the floor extending through the location of the visible marker corresponding to the radiographic fiducial.

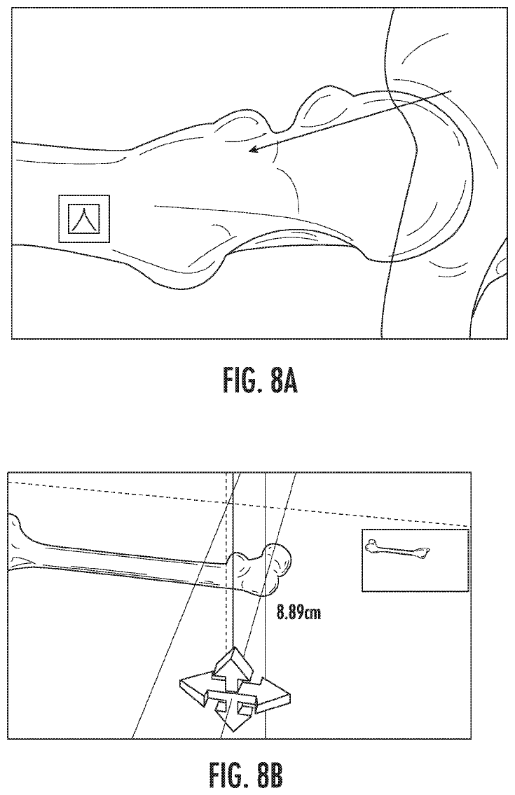

The desired target point is then chosen on the lateral image (manually or automatically) as is shown in FIG. 8A and a corresponding virtual line drawn as shown in FIG. 8B. FIG. 8A illustrates a lateral fluoroscopic image of the left hip including radiographic fiducial. Desired position indicated by tip of blue arrow. FIG. 8B illustrates an augmented view of an anatomical femur model showing a virtual line extending through the original location of the fiducial and the desired location at the tip of the greater trochanter.

In this example, the target position on the lateral radiograph intersects the position identified on the AP radiograph. This is a requirement if the user desires to visualize two intersecting lines as a starting point if the lines are drawn independently. However, it is just as possible to define this condition in the software on the HMD. In this case, two axes are defined from one plane (the AP image), and only a third axis is required from the orthogonal image. This would ensure that the two lines always intersect at the point of interest. This is likely the advantageous method of linking the fluoroscopic image to the world frame; however, two independent virtual lines is described here for simplicity purposes.

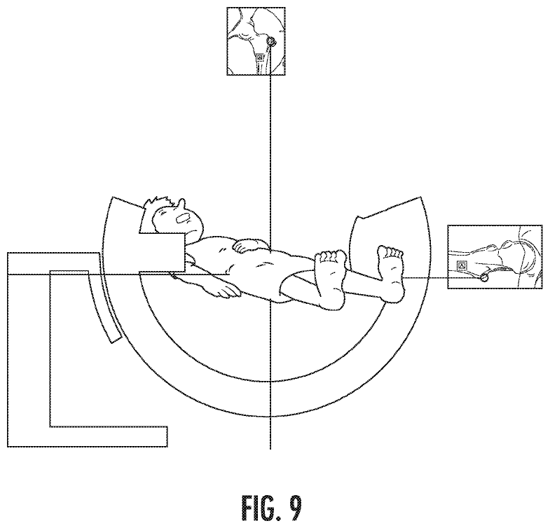

Once the second target position is identified and the scaling and translations have occurred then the user should visualize a virtual line intersecting the point of interest on the AP image and a line extending parallel to the floor intersecting the point of interest on the lateral image as is seen in FIG. 9. FIG. 9 illustrates a schematic diagram of virtual lines in the world frame of reference intersecting the desired points on the AP and lateral fluoroscopic images.

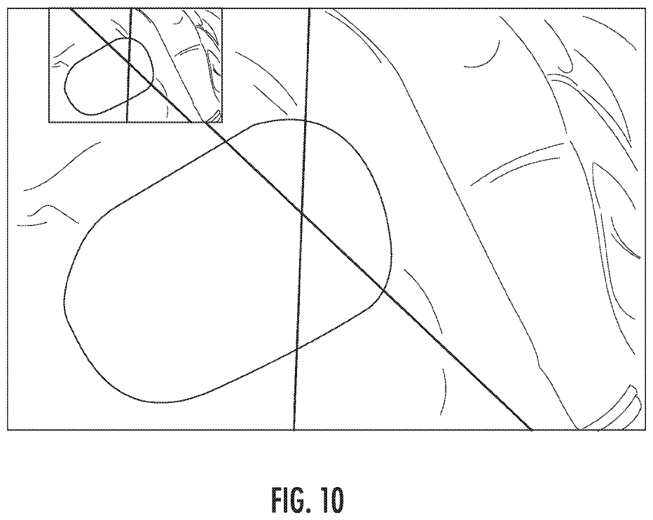

At this point, virtual lines have been drawn intersecting the points of interest in both the AP plane and the lateral plane. The intersection of these two lines then defines the desired point in 3D space that is the desired target as seen in FIG. 10. FIG. 10 illustrates an augmented view showing lines intersecting at the target point within the body.

The previous example of finding the starting point for intramedullary nailing of the femur is used to describe the proposed method of registering the fluoroscopic image to the spatial map. However, this method can be applied for a wide variety of other cases in surgery and medicine. For example, for just this one procedure of intramedullary nailing of the femur, this system could be used for guidance for the following steps:
1) Placing a guide wire to the desired entry point on the femur
2) Confirming correct length of the intramedullary nail and position of the nail within in the femur
3) Correctly aligning the hip screw along the desired trajectory of the femoral neck and into the femoral head
4) Ensuring the correct length of the hip screw
5) Placing distal interlocking screws
6) Ensuring correct length, alignment, and rotation of the limb

### Description of a Prototype of the System Using Existing Hardware:

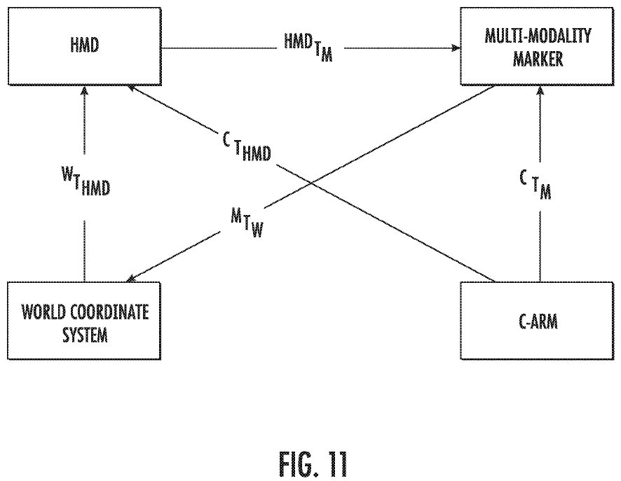

The described system includes three components that must exhibit certain characteristics to enable on-the-fly AR guidance: a mixed-modality fiducial, a C-Arm X-Ray imaging system, and an optical see-through HMD. Based on these components, the spatial relations that need to be estimated in order to enable real-time AR guidance are shown in FIG. 11. FIG. 11 illustrates a schematic diagram of spatial transformations for on-the-fly AR solutions. Put concisely, the present invention is directed to recovering the transformation
^{C}T_{HMD}(t) that propagates information from the C-arm to HMD coordinate system while the surgeon moves over time t. To this end, the following transformations are estimated:
^{C}T_{M}: Extrinsic calibration of the C-Arm to the multi-modality marker domain.
^{M}T_{W}: Describes the mapping from the multi-modality marker to the world coordinate system.
^{HMD}T_{M}: Transformation describing the relation between the HMD and the multi-modality marker coordinate system.
^{W}T_{HMD}: Transformation from the world to the HMD domain. Once these relations are known, annotations in an intra-operatively acquired X-Ray image can be propagated to and visualized by the HMD, which provides support for placement of wires and screws in orthopaedic interventions. The transformation needed is given by:

$${}_{}^{C}{T}{}_{HMD}^{} ( t ) = {}_{}^{W}{T}{}_{HMD}^{} ( t ) \underbrace{{(}^{W} {T}_{HMD}^{- 1} ( {t}_{0} ) {}_{}^{HMD}{T}{}_{M}^{- 1} ( {t}_{0} ) ) {}_{}^{C}{T}{}_{M}^{} ( {t}_{0} )} , \qquad ( 1 )$$

where t₀ denotes the time of calibration, e. g. directly after repositioning of the C-Arm, suggesting that ^{C}T_{W} is constant as long as the C-Arm remains in place. For brevity of notation, the time dependence of the transformations is omitted whenever they are clear or unimportant. Detailed information on the system components and how they are used to estimate aforementioned transformations is provided herein.

The key component of this particular method of image to world registration using a world spatial map is a multi-modality marker that can be detected using a C-Arm as well as the HMD using X-Ray and RGB imaging devices, respectively. As the shape and size of the multi-modality marker is precisely known in 3D, estimation of both transforms ^{C}T_{M} and ^{HMD}T_{M} is possible in a straightforward manner if the marker can be detected in the 2D images. In this prototype example, ARToolKit is used for marker detection and calibration; however, it is not a necessary component of the system as other detection and calibration algorithms can be used.

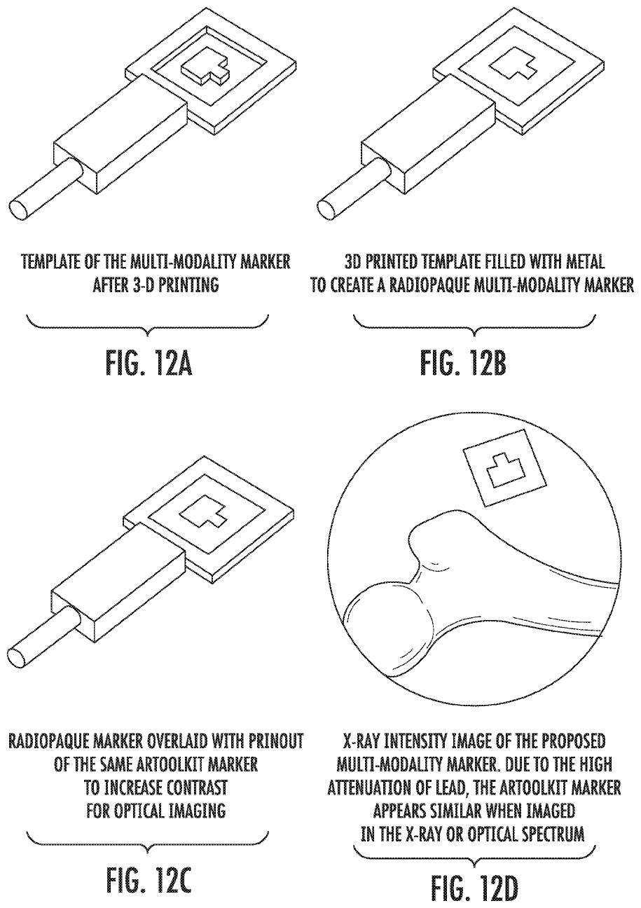

FIGS. 12A-12D illustrate image views of steps in the creation of the multi-modality marker. The marker needs to be well discernible when imaged using the optical and X-Ray spectrum. To this end, the template of a conventional ARToolKit marker is printed as shown in FIG. 12A that serves as the housing for the multi-modality marker. FIG. 12A illustrates a template of the multi-modality marker after 3D printing. Then, a metal inlay (solder wire 60n40 SnnPb) that strongly attenuates X-Ray radiation is machined, see FIG. 12B. FIG. 12B illustrates a 3D printed template filled with metal to create a radiopaque multi-modality maker. After covering the metal with a paper printout of the same ARToolKit marker as shown in FIG. 12C, the marker is equally well visible in the X-Ray spectrum as well as RGB images due to the high attenuation of lead as can be seen in FIG. 12D. FIG. 12C illustrates a radiopaque marker overlaid with a printout of the same, and FIG. 12D illustrates an X-Ray intensity image of the proposed multi-modality marker. This is very convenient, as the same detection and calibration pipeline readily provided by ARToolKit can be used for both images. Due to the high attenuation of lead, the ARToolKit marker appears similar when imaged in the X-Ray or optical spectrum.

It is worth mentioning that the underlying vision-based tracking method in ARToolKit is designed for reflection and not for transmission imaging which can be problematic in two ways. First, ARToolKit assumes 2D markers suggesting that the metal inlay must be sufficiently thin in order not to violate this assumption. Second, a printed marker imaged with an RGB camera perfectly occludes the scene behind it and is, thus, very well visible. For transmission imaging, however, this is not necessarily the case as all structures along a given ray contribute to the intensity at the corresponding detector pixel. If other strong edges are present close to this hybrid marker, detection and hence calibration may fail. In this prototype example, to address both problems simultaneously digital subtraction is used, a concept that is well known from angiography. Two X-Ray images are acquired using the same acquisition parameters and C-Arm pose both with and without the multi-modality marker introduced into the X-Ray beam. Logarithmic subtraction then yields an image that, ideally, only shows the multi-modality marker and lends itself well to marker detection and calibration using the ARToolKit pipeline. Moreover, subtraction imaging allows for the use of very thin metal inlays as subtraction artificially increases the contrast achieved by attenuation only. While the subtraction image is used for processing, the surgeon is shown the fluoroscopy image without any multi-modality marker obstructing the scene.

This system has the substantial advantage that, in contrast to many previous systems, it does not require any modifications to commercially available C-Arm fluoroscopy systems. The only requirement is that images acquired during the intervention can be accessed directly such that geometric calibration is possible. In the prototype example described herein, a Siemens ARCADIS Orbic 3D (Siemens Healthcare GmbH, Forchheim, Germany) is used to acquire fluoroscopy images and a frame grabber (Epiphan Systems Inc, Palo Alto, Calif.) paired with a streaming server to send them via a wireless local network to the HMD. While extrinsic calibration of the C-Arm system is possible using the multi-modality marker as detailed herein, the intrinsic parameters of the C-Arm, potentially at multiple poses, are estimated in a one-time offline calibration using a radiopaque checkerboard.

Once the extrinsic and intrinsic parameters are determined, the 3D source and detector pixel positions can be computed in the coordinate system of the multi-modality marker. This is beneficial, as simple point annotations on the fluoroscopy image now map to lines in 3D space that represent the X-Ray beam emerging from the source to the respective detector pixel. These objects, however, cannot yet be visualized at a meaningful position as the spatial relation of the C-Arm to the HMD is unknown. The multi-modality marker enabling calibration must be imaged simultaneously by the C-Arm system and the RGB camera on the HMD to enable meaningful visualization in an AR environment. This process will be discussed in greater detail below.

The optical see-through HMD is an essential component of the proposed system as it needs to recover its pose with respect to the world coordinate system at all times, acquire and process optical images of the multi-modality marker, allow for interaction of the surgeon with the supplied X-Ray image, combine and process the information provided by the surgeon and the C-Arm, and provide real-time AR visualization for guidance. In this exemplary embodiment of the present invention, the Microsoft HoloLens (Microsoft Corporation, Redmond, Wash.) is used as the optical see-through HMD, as its performance compared favorably to other commercially available devices.

Similar to the pose estimation for the C-Arm, the pose of the HMD is estimated with respect to the multi-modality marker ^{HMD}T_{M}. In order to allow for calibration of the C-Arm to the HMD, the images of the marker used to retrieve ^{C}T_{M} and ^{HMD}T_{M} for the (a) AR environment with a single C-Arm view. (b) AR environment when two C-Arm views are used.

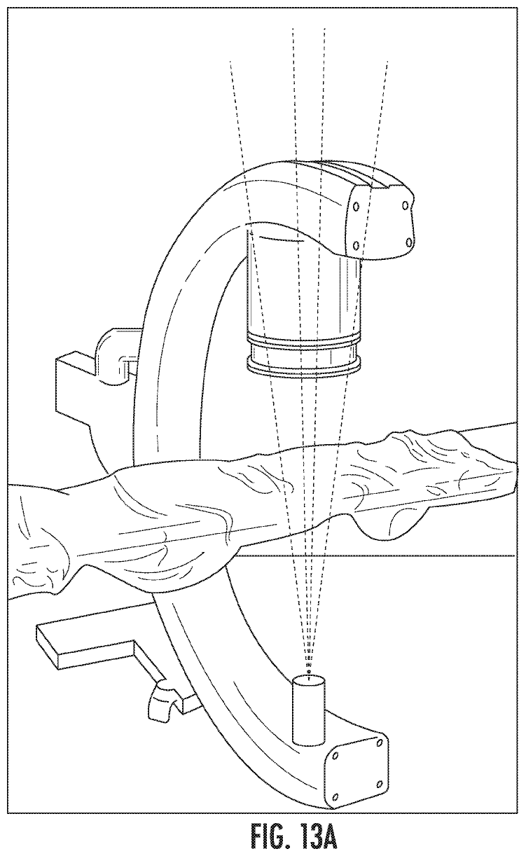

FIGS. 13A and 13B illustrate image views of source position of the C-Arm shown as a cylinder and virtual lines that arise from annotations in the fluoroscopy image. The C-Arm and the HMD, respectively, must be acquired with the marker at the same position. If the multi-modality marker is hand-held, the images should ideally be acquired at the same time to. The HoloLens is equipped with an RGB camera that used to acquire an optical image of the multi-modality marker and estimate ^{HMD}T_{M} using ARToolKit. In principle, these two transformations are sufficient for AR visualization but the system would not be appropriate, if the surgeon wearing the HMD moves, the spatial relation ^{HMD}T_{M} changes.

As limiting the surgeons movements is not feasible, updating ^{HMD}T_{M}(t) over time may seem like an alternative. However, is impracticable as it would require the multi-modality marker to remain at the same position, potentially close to the operating field. While updating ^{HMD}T_{M}(t) over time seems complicated, recovering ^{HMD}T_{W}(t), the pose of the HMD with respect to the world coordinate system, is readily available from the HoloLens headset and is estimated via simultaneous localization and mapping (SLAM). Consequently, rather than directly calibrating the C-Arm to the HMD, the C-Arm is calibrated to the world spatial map to retrieve ^{C}T_{W} that is constant if the C-Arm is not repositioned.

In order to use the system for guidance, key points must be identified in the X-Ray images. Intra-operative fluoroscopy images are streamed from the C-Arm to the HMD and visualized using a virtual monitor. The surgeon can annotate anatomical landmarks in the X-Ray image by hovering the HoloLens cursor over the structure and performing the air tap gesture. In 3D space, these points must lie on the line connecting the C-Arm source position and the detector point that can be visualized to guide the surgeon using the spatial relation in Eq. 1. An exemplary scene of the proposed AR environment is provided in FIGS. 13A and 13B. Guidance rays are visualized as semi-transparent lines with a thickness of 1 mm while the C-Arm source position is displayed as a cylinder. The association from annotated landmarks in the X-Ray image to 3D virtual lines is achieved via color coding.

It is worth mentioning that the proposed system allows for the use of two or more C-Arm poses simultaneously. When two views are used, the same anatomical landmark can be annotated in both fluoroscopy images allowing for stereo reconstruction of the landmark's 3D position. In this case, a virtual sphere is shown in the AR environment at the position of the triangulated 3D point, shown in FIG. 13B. Furthermore, the interaction allows for the selection of two points in the same X-Ray image that define a line. This line is then visualized as a plane in the AR environment. An additional line in a second X-Ray image can be annotated resulting in a second plane. The intersection of these two planes in the AR space can be visualized by the surgeon and followed as a trajectory.

Once the HMD is connected to the C-Arm, only very few steps for obtaining AR guidance are needed. For each C-Arm pose, the surgeon has to: 1. Position the C-Arm using the integrated laser cross-hair such that the target anatomy will be visible in fluoroscopy. 2. Introduce the multi-modality marker in the C-Arm field of view and also visible in the RGB camera of the HMD. If the fiducial is recognized by the HMD, an overlay will be shown. Turning the head such that the marker is visible to the eye in straight gaze is usually sufficient to achieve marker detection. 3. Calibrate the system by use of a voice command (“Lock”) and simultaneously acquiring an X-Ray image with the marker visible in both modalities. This procedure defines to and thus ^{C}T_{W} in Eq. 1. Note that in the current system, a second X-Ray images needs to be acquired for subtraction but the marker can also be removed from the scene in other embodiments. 4. Annotate the anatomical landmarks to be targeted in the fluoroscopy image.

Performing the aforementioned steps yields virtual 3D lines that may provide sufficient guidance in some cases, however, the exact position of the landmark on this line remains ambiguous. If the true 3D position of the landmark is needed, the above steps can be repeated for another C-Arm pose.

### Advantages of System:

1) Requires no external hardware or software other than spatial mapping capable HMD (no interface with c-arm, ct, medical records, etc) (for offline system)
2) Does not require mixed modality AR marker to be in the line of site of the user after initial world locked point is chosen
3) Does not require preoperative imaging or data
4) No setup or initialization required
5) Consistent with conventional workflow
6) Intuitive to use

### Broader Concepts and Use:

As explained above, this method is at its core a new surgical registration system. Prior systems have linked an image frame of reference to a tracker “world” frame of reference or an image frame of reference to a C-arm based infrared point cloud; however, this is the first description of registration of an image frame of reference to a “markerless” world spatial map. The above example provides the basic concept of how it may be used and the method translation; however, this new method of registration can be used beyond this example case.

Ultimately any version of this registration method must have the following 2 fundamental components: 1) The ability to select a unique “locked” position on the world spatial map. 2) The ability to recognize this unique position in an acquired image. The above examples discuss two main ways of achieving the first component. Either an automatically tracked marker is recognized and then locked to the world spatial map, or the hand, head, or eyes are tracked and used to localize a unique point in the world spatial map. The methods of achieving the second component are much broader and which method is used depends upon a wide variety of factors.

The first consideration, which was discussed above, is whether the system is “offline” or “online”. An “offline” system using only the HMD without any modification of the imaging machine or connection to the imagine machine is limited by what it can detect in the camera view of the monitor being used to display the images. It is more difficult to perform complex image processing on this “image of an image” than it is to perform such processing on the actual data collected from the imaging machine. In this case, the more obvious the “world locked” point (marker, end of tool, landmark, etc.) is in the image, the easier it is to detect. This is why an X-Ray visible augmented reality marker is suitable for this scenario. It is easily recognizable on the monitor and its properties allow for “processing” of the image without dealing with data output from the machine itself. Theoretically, even complex image processing could be performed on the “image of an image” in the offline system; however, such complex processing is much more readily achievable in an “online” system and is why an “online” system was chosen for the prototype described above.

Additionally, an “online” system allows for all of the image processing available to other registration and navigation systems to be combined with the ability to link this information to the world spatial map. Therefore, points on common surgical tools can be selected for “world locked” points and their radiographic projections can be automatically recognized via various methods including machine learning algorithms. Furthermore, in an “online” system, depth information can be teased out of 2D radiographic images, and depth of structures within these images can be compared to the perceived depth of the radiographic fiducial. With the position of the fiducial known and “world locked”, this depth information can be used to localize structures in 3D space by using a single 2D fluoroscopic image. This process is made simpler when preoperative imaging is available, whether 2D or 3D. Such imaging can give clues as to the actual size of the anatomical structures, which along with the physical properties of the fiducial and the 2D image, can allow for attainment of depth information in these 2D images and for localization of anatomical structures in 3D space. Furthermore, 2D to 3D registration methods can be performed on any 2D imaging obtained so that a 3D model could be locked to its correct location in the spatial map based upon a 2D image alone.

Image “Mapping” and “Stitching” of Non-Overlapping Areas:

Registering every acquired image to a world spatial map allows for creation of an “image map” that can be updated with each new image taken. Each image is uniquely mapped to a point in space at which the mixed-modality AR marker was positioned at the time the image was acquired. As well, the orientation of that image (plane of the X-Ray beam in regard to the AR marker) is also known and can be displayed on the image map. Therefore, for every pose of the C-arm and/or new position of the fiducial, a new image is added to the image map. In this way, every new image acquired adds additional information to the image map that can be used by the surgeon. Traditionally, the majority of fluoroscopic images are “wasted”. That is, the image is viewed once and then discarded as a new image is obtained. Sometimes the original image contains pertinent information to the surgeon, but many times images are taken in an attempt to obtain the desired image with the information valuable to the surgeon (such as a perfect lateral radiograph of the ankle, or a “perfect circle” view of the distal interlocks of an intramedullary nail). With the proposed system of image mapping, every image acquired can be added to the map to create a “bigger picture” that the surgeon can use. Furthermore, as each image is linked to all of the others, spatial orientation is known between anatomical structures within each image. This allows for length, alignment, and rotation to be determined in an extremity based upon images of non-overlapping regions, such as an image of the femoral head and the knee. Lastly, an image map allows surgeons to “interpolate” between images that have been obtained. For example, if an image exists of the femoral head and another image exists of the knee, the surgeon can imagine from the image map where the shaft of the femur might be in 3D space.

3D Imaging:

These principles do not only apply to 2D intraoperative imaging, but 3D as well, such as cone beam CT and O-arm scanning. Currently, many navigation systems using 3D imaging employ optical trackers to register CT scans to the body on the surgical table. In order to use an HMD for visualization, the HMD must also be tracked with optical trackers. Other navigation systems using 3D imaging employ depth cameras fixed to the cone beam CT (CBCT) scanner itself. These cameras then are able to link the location of the anatomical structure of interest to a 3D point cloud generated by the information sensed in a depth camera. However, these systems require that the hands/tools be in the view of the depth camera in order to be displayed in the point cloud. Furthermore, the CBCT scanner must stay at the same location and pose at which it was located when it first acquired the scan. Using image to world spatial map registration, CT imaging can be “locked” to a position in the world rather than to an optical tracker or a point cloud centered on the CBCT scanner itself. For fixed anatomy (such as the pelvis or spine), this registration method has an advantage over optical tracking systems in that the body and the HMD would not have to be optically tracked. It holds great benefit over CBCT image to CBCT machine based point cloud registration in that the 3D anatomical information from the scan can be locked to its correct position in the world. The CBCT machine can then be moved away from the table or to a different location or pose while the 3D information is still visible at the correct location as displayed by the HMD.

One method for use of the mixed modality marker for 3D imaging is place a marker onto a patient before 3D imaging is obtained and it is present on the surface of the patient during scanning, then it can later be used to overlay that same imaging back onto the patient. This marker on the surface of the patient can then be “world locked” to fix the 3D imaging in place so that the marker does not need to be continuously tracked by the HMD to display the 3D visualization.

### Method of Image to World Registration Using a World Spatial Map and Registration of Multiple Overlapping World Spatial Maps.

The following is another example of a method for image to world registration using a world a spatial map. However, this method uses overlapping world spatial maps and inside out tracking to register the image to the world coordinate system. Again, this is a specific method of the more broadly claimed image to world registration using a world spatial map concept.

The inside-out tracking paradigm, core of the proposed concept, is driven by the observation that all relevant entities (surgeon, patient, and C-arm) are positioned relative to the same environment, the “world coordinate system” which is acquired with world spatial mapping. For intra-operative visualization of 3D volumes overlaid with the patient, we seek to dynamically recover the transformation describing the mapping from the surgeon's eyes to the 3D image volume. In the following equation (Equation X), to describes the time of pre to intraoperative image registration while t is the current time point. The spatial relations required to dynamically estimate ^{S}T_{V} are explained in the remainder of this section and visualized in Figure X.

$${}_{}^{S}{T}{}_{V}^{} ( t ) = {}_{}^{S}{T}{}_{W}^{} \underbrace{{(}^{T} {T}_{W}^{- 1} ( {t}_{0} ) {}_{}^{T}{T}{}_{C}^{} ( {t}_{0} ) ) {}_{}^{V}{T}{}_{C}^{- 1}} ,$$

^{W}T_{S/T}. The transformations ^{W}T_{S/T} are estimated using Simultaneous Localization and Mapping (SLAM) thereby incrementally constructing a map of the environment, i.e. the world coordinate system or the world spatial map. Exemplarily for the surgeon SLAM solves

$${}_{}^{W}{T}{}_{S}^{} ( t ) = {argmin}_{{}_{}^{W}{\hat{T}}{}_{S}^{}} d ( {f}_{W} ( P {}_{}^{W}{\hat{T}}{}_{S}^{} ( t ) {x}_{S} ( t ) ) , {f}_{S} ( t ) ) ,$$

where fₛ(T) are features in the image at time t, xs(t) are the 3D locations of these feature estimates either via depth sensors or stereo, P is the projection operator, and d(⋅, ⋅) is the feature similarity to be optimized. A key innovation of this work is the inside-out SLAM-based tracking of the C-arm w.r.t. the environment map by means of an additional tracker rigidly attached to the C-shaped gantry. This becomes possible if both trackers observe partially overlapping parts of the environment, i. e. a feature rich and temporally stable area of the environment. This suggests, that the cameras on the C-arm tracker need to face the room rather than the patient.

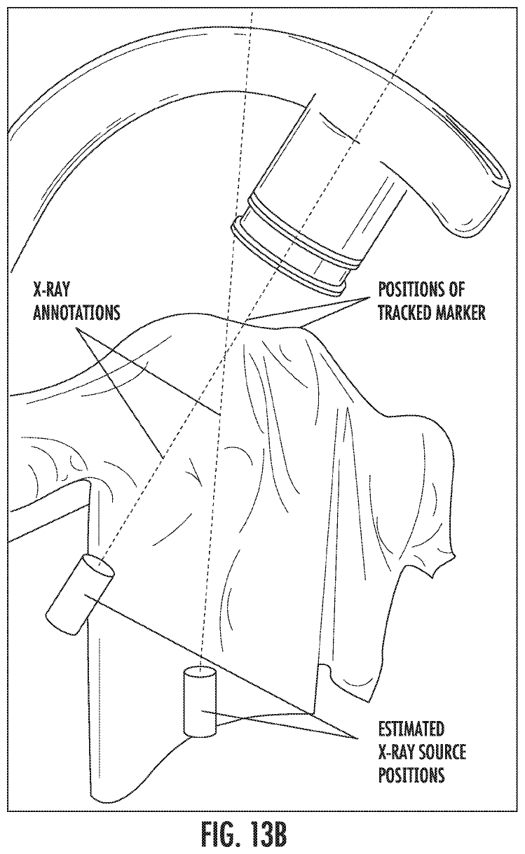

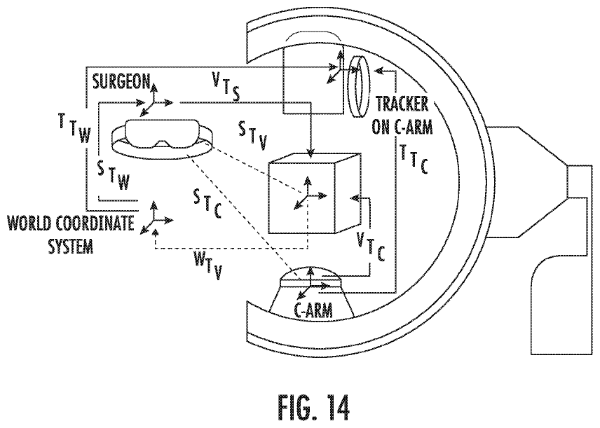

FIG. 14 illustrates a schematic diagram phantoms used in studies assessing the performance of the system in a surgery-like scenario. ^{T}T_{C}: The tracker is rigidly mounted on the C-arm gantry suggesting that one-time offline calibration is possible. Because the X-ray and tracker cameras have no overlap, methods based on multi-modal patterns fail. However, if poses of both cameras with respect to the environment and the imaging volume, respectively, are known or can be estimated, Hand-Eye calibration is feasible. Put concisely, a rigid transform ^{T}T_{C} is estimated such that A(tᵢ)^{T}T_{C}=^{T}T_{C}B(tᵢ), where (A/B)(tᵢ) is the relative pose between subsequent poses at time i, i+1 of the tracker and the C-arm, respectively. Poses of the C-arm ^{V}T_{C}(Tᵢ) are known because a prototype of the present invention uses a cone-beam CT (CBCT) enabled C-arm with pre-calibrated circular source trajectory, such that several poses ^{V}T_{C} are known. During one sweep, the poses of the tracker are estimated to be ^{W}T_{T}(tᵢ). Finally, ^{T}T_{C} is recovered and thus ^{W}T_{C}.

^{V}T_{C}: To close the loop by calibrating the patient to the environment, ^{V}T_{C} is estimated, describing the transformation from 3D image volumes to an intra-operatively acquired X-ray image. For preoperative date, ^{V}T_{C} can be estimated via image based 3D/2D registration. If the C-arm is CBCT capable and the 3D volume is acquired intra-procedurally, ^{V}T_{C} is known and can be defined as one of the pre-calibrated C-arm poses on the source trajectory, e.g. the first one. Once ^{V}T_{C} is known the volumetric images are calibrated to the room via ^{W}T_{V}=^{W}T_{T}(t₀)^{T}T_{C}^{V}T_{C}⁻¹(t₀), where to denotes the time of calibration.

In summary, this method utilizes a C-arm with a rigidly mounted tracker capable of creating a world spatial map, to which the position of the C-arm is known. A second tracker, creating its own world spatial map, is including on the HMD and thus knows the position of the HMD to the world spatial map. These two maps overlap and are correlated so that the position of C-arm is known to the HMD via the relation of the spatial maps.

Another aspect and embodiment of the present invention is directed to a “mixed modality” fiducial as discussed herein with all properties necessary for functioning as so described and with additional unique properties so as to make possible other features for an image to world spatial map augmented reality surgical system. The invention is at a minimum an augmented reality marker with the following fundamental component features: 1. The AR marker is recognizable by a camera and virtual information can be mapped to it. 2. The AR marker has a radiographic projection that is visually detectable in fluoroscopic/radiographic images.

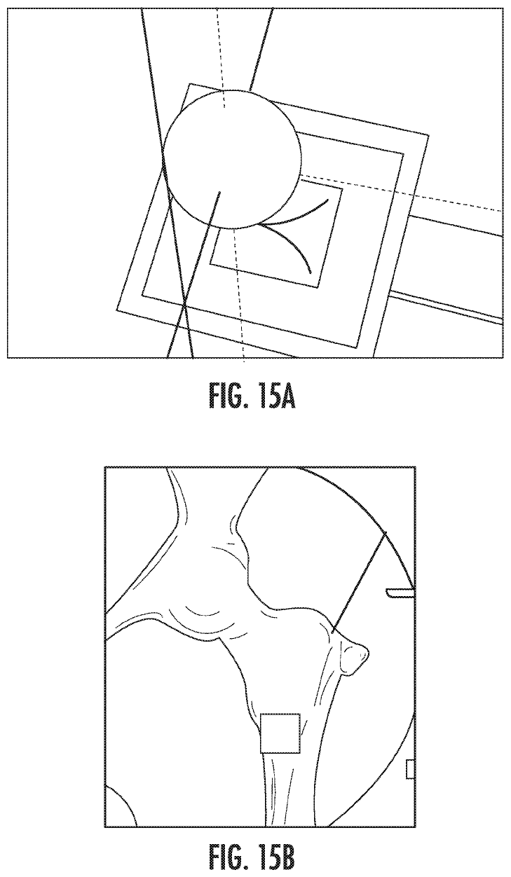

FIG. 15A illustrates a perspective view of an augmented reality marker, according to an embodiment of the present invention, and FIG. 15B illustrates an image view of a radiographic projection of the augmented reality marker as it would appear in a fluoroscopic image. FIG. 15A shows the augmented reality marker's visibility to the camera and the ability for virtual information to be mapped to it. FIG. 15B shows a theoretical radiographic projection of the same augmented reality marker as it would appear in a fluoroscopic image. Note it is easily recognizable in the fluoroscopic image. These two features include the minimal components of the invention and an AR marker with these minimal features is in and of itself novel and capable for use in an image to world spatial map registration augmented reality surgical system. The next level of complexity is a feature that links positional information between the image frame of reference and the world frame of reference. This would mean that one could deduce the orientation of the augmented reality marker as it was positioned when the radiographic image was obtained. This additional feature is summarized in the following: 3. The orientation of the AR marker in the world frame of reference is linked to its orientation in the image frame of reference.

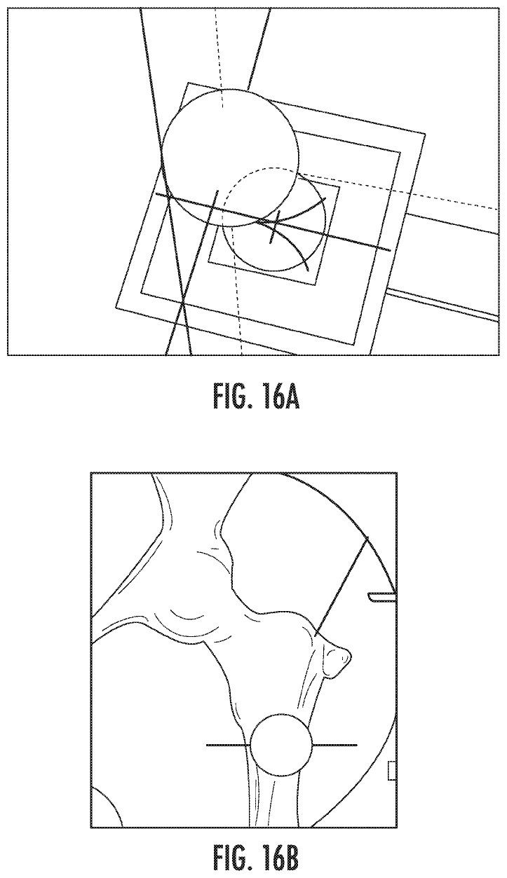

The additional feature of the orientation of the AR marker in the frame would look like the embodiment of FIGS. 16A and 16B. FIG. 16A illustrates a perspective view of an augmented reality marker recognizable to camera with virtual information mapped to it. Note also the orientation of the marker denoted by the gray circle and line. FIG. 16B illustrates an image view of a theoretical radiographic projection of the same augmented reality marker as it would appear in a fluoroscopic image. Note its orientation is easily recognizable in the fluoroscopic image.

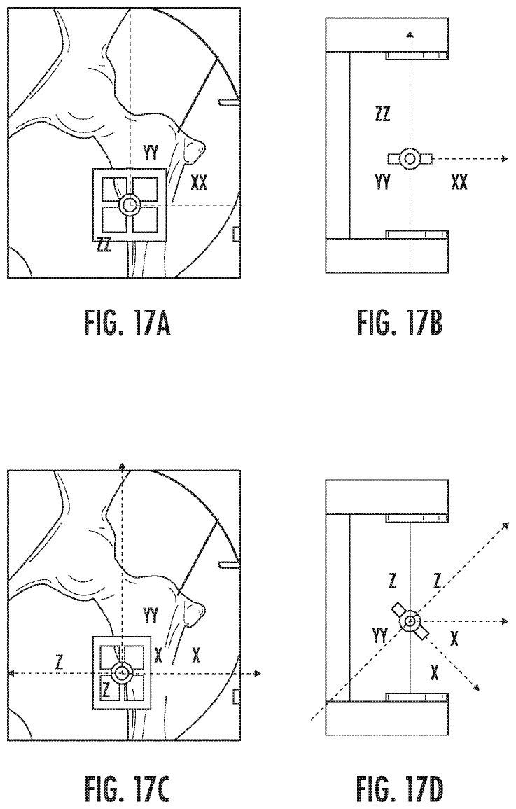

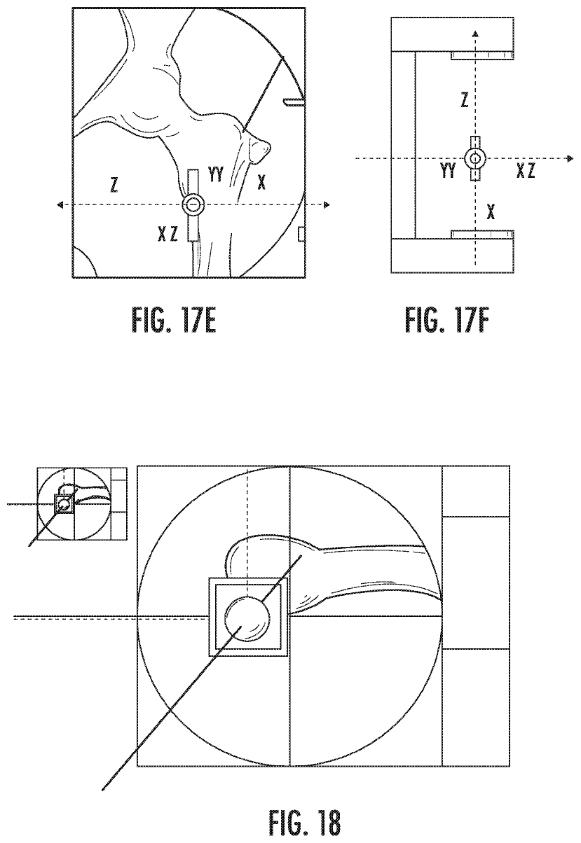

Another element of the present invention allows for the orientation and relation of the X-ray beam to the physical AR marker to be deduced from the projectional appearance of the AR marker in the radiographic image. This is an important feature as it could ensure that translations in the image frame and the world frame are occurring in the same plane. For example, when the C-arm is oriented for a direct AP image (beam perpendicular to the floor) the projectional plane upon which translations can be made will be parallel to the floor. If translations are to be made in the world frame based upon this AP image, then these translations can only be accurately performed in the same plane of the projectional image that is parallel to the floor. FIGS. 17A-17F illustrate image and schematic views of the projectional appearance of the AR marker on the radiograph shows how the x-ray beam impacted it in the physical world frame. FIGS. 17A and 17B illustrate the AR target perpendicular to the x-ray beam, both the image coordinate system and the world coordinate system are aligned. FIGS. 17C and 17D illustrate the AR target tilted 45 degrees in relation to the x-ray beam, the world coordinate system has rotated 45 degrees in relation to the image coordinate system. FIGS. 17D and 17F illustrate the AR target tilted 90 degrees in relation to the x-ray beam, the world coordinate system has rotated a full 90 degrees in relation to the image coordinate system.

In an image to world spatial map registration AR surgical system, if the coordinate system of the image and the world are aligned, then the image must simply be appropriately translated in order to correlate points on the image to the world frame.

In the previous example, the AR marker is being rotated in relation to a fixed x-ray beam; however, the orientation information is also maintained when the C-arm and x-ray beam are moved in relation to a static AR marker. However, different from the above example, when the beam is rotated, the actual fluoroscopic image will change as well as the appearance of the AR marker in the image. FIG. 18 illustrates a perspective view of a fluoroscopic image including an X-Ray visible AR marker with virtual information mapped to the radiographic projection. The radiographic projection of the AR marker is also recognizable by the camera so that virtual information can be mapped to it. This means that the radiographic projection of the AR marker acts as an AR marker itself, as illustrated in FIG. 18. Further, the image coordinate system can be automatically translated to the world coordinate system.

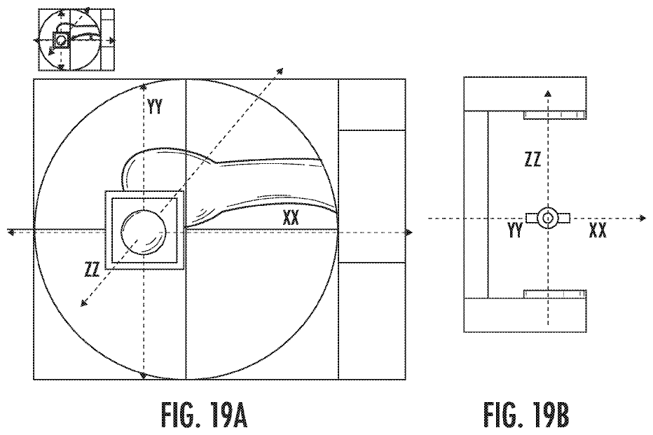

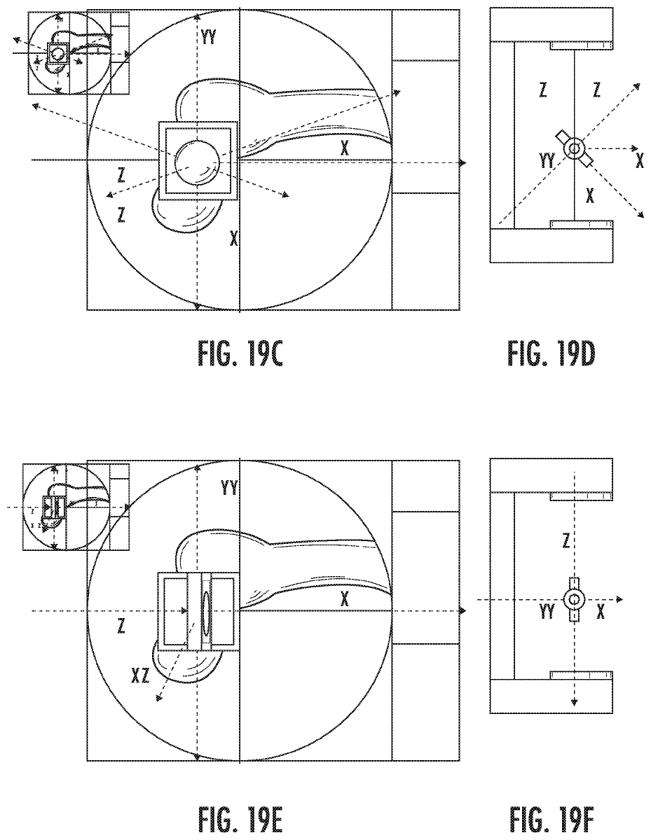

When the target is recognized, its size on the projectional image is also recognized. The actual physical size of the AR marker is known. The orientation between coordinate systems (when not aligned) can be automatically deduced from the recognition of the radiographic projection of the AR marker by the camera, as illustrated in FIGS. 19A-19F. FIGS. 19A-19F illustrate image and schematic views of an embodiment of the present invention. FIGS. 19A and 19B show the image coordinate system and the world coordinate system are aligned as well as the coordinate system of the radiographic projection of the AR marker. In FIGS. 19C and 19D the world coordinate system is rotated 45 degrees from the image coordinate system. In FIGS. 19E and 19F the world coordinate system is rotated 90 degrees from the image coordinate system; however, the coordinate system of the radiographic projection of the AR marker is still aligned with the world system.

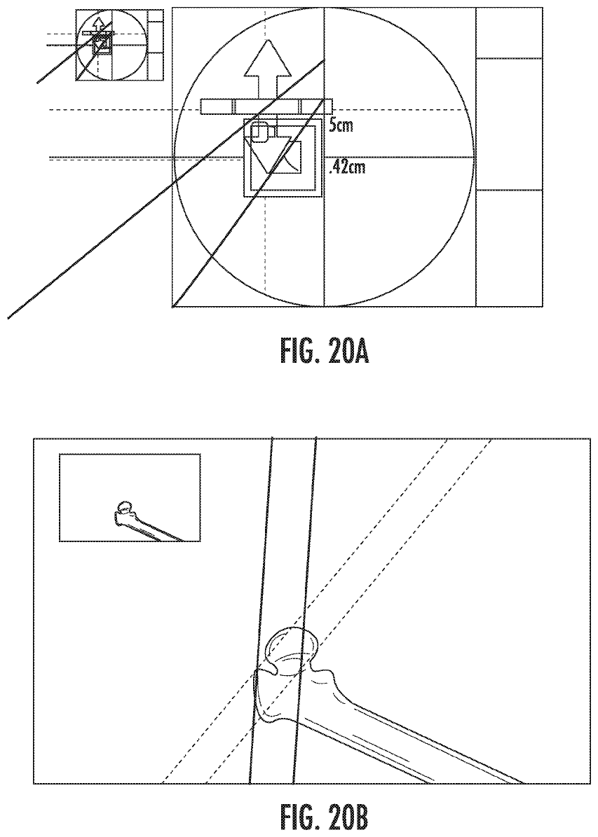

Virtual information can be automatically superimposed onto the physical monitor. In procedures using fluoroscopic guidance, the fluoroscopic image is the “road map” driving how to place needles, wires, implants, etc. in the body. The ability to select points, annotate and measure on the physical screen intra-operatively is a valuable feature for surgeons. Furthermore, having these selections, annotations, and measurements visible in the world frame is also incredibly valuable. This allows the physical screen to become a “touch pad” with which the surgeon can interact. Any interactions on this “touch screen” can also simultaneously be displayed in the world frame of reference, as illustrated in FIGS. 20A and 20B. FIGS. 20A and 20B illustrate image views of exemplary images with which the physician can interact. FIG. 20A illustrates a fluoroscopic image including the radiographic projection of the AR marker with virtual information mapped to it. The line intersects perpendicularly with the center of the radiographic projection of the AR marker. The arrows demonstrate a simple manner in which to “interact” with the screen. The line intersects a new desired point on the fluoroscopic image that was selected by sliding the arrows along the screen. FIG. 20B illustrates an augmented view of the world frame (in this case an anatomic femur model), the red line represents the original location that was perpendicular to the AR marker. The blue line intersects the new point that was chosen in the fluoroscopic image by “interacting” with the screen.

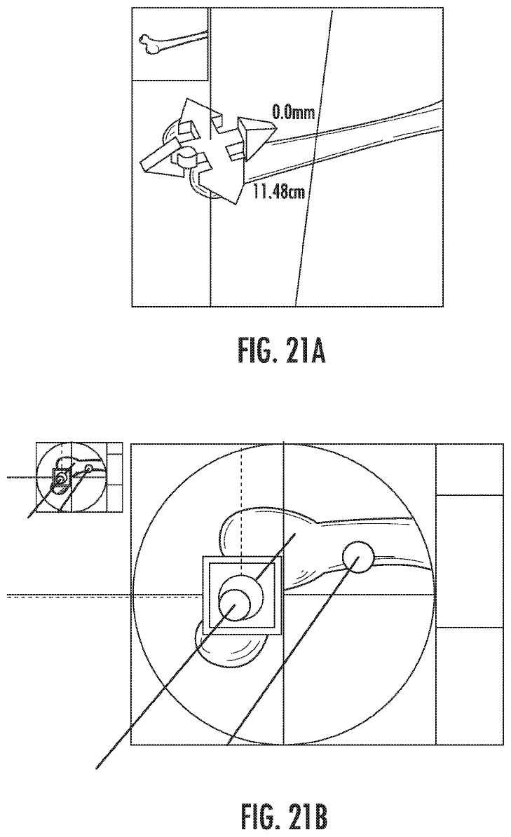

Note, this procedure can be done in reverse well. For example, the interaction can take place with the virtual information in the world frame of reference and the corresponding translation can be viewed in the image frame of reference, as illustrated in FIGS. 21A and 21B. FIGS. 21A and 21B illustrate image views of exemplary images with which the physician can interact. In FIG. 21A, an augmented view of the world frame (in this case an anatomic femur model), the blue line represents the original location that was perpendicular to the AR marker. The red line is the new location in the world frame that was selected by interacting with the virtual information (via green arrow sliders). In FIG. 21B, the blue circle and line represent the location of the radiographic projection of the AR marker which correlates to the blue line in FIG. 21A. The red circle and line represent the point in the fluoroscopic image correlating to the red line FIG. 21A encompassing the translation that was completed in the world frame of reference.

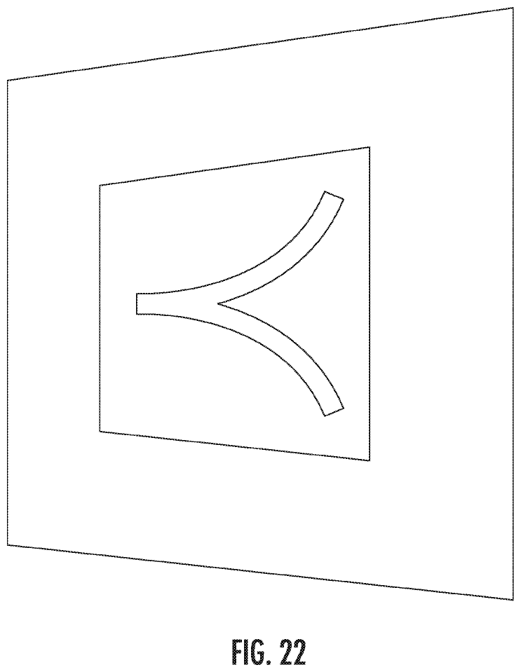

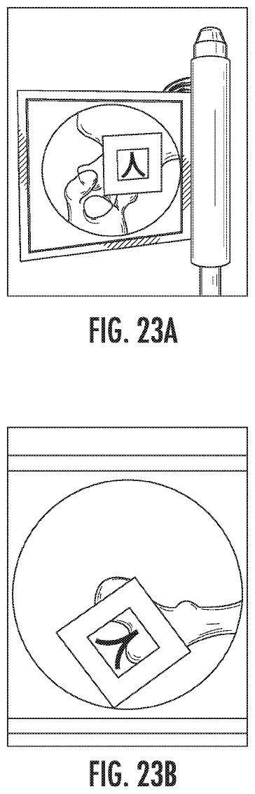

Such an AR marker could be made in a variety of different ways. One simple method is to build a marker out of radiopaque material so that the black portion of the traditional marker is radiopaque while the white portion is radiotranslucent as in FIGS. 22 and 23A and 23B. This could be achieved by cutting the pattern from a piece of sheet metal. FIG. 22 illustrates a perspective view of a radiovisible Augmented Reality Marker. The dark portions are filled with lead plate while the light portions are filled with foam board. FIGS. 23A and 23B illustrate image views of fluoroscopic images demonstrating the radiographic projection of the lead AR marker.

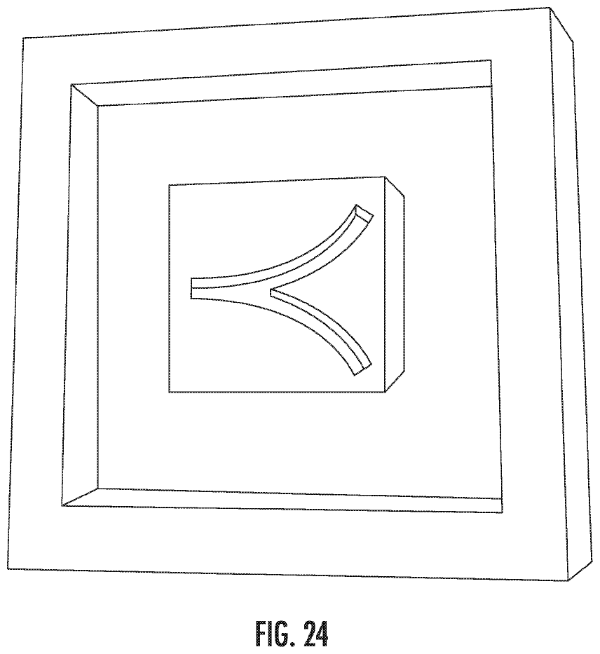

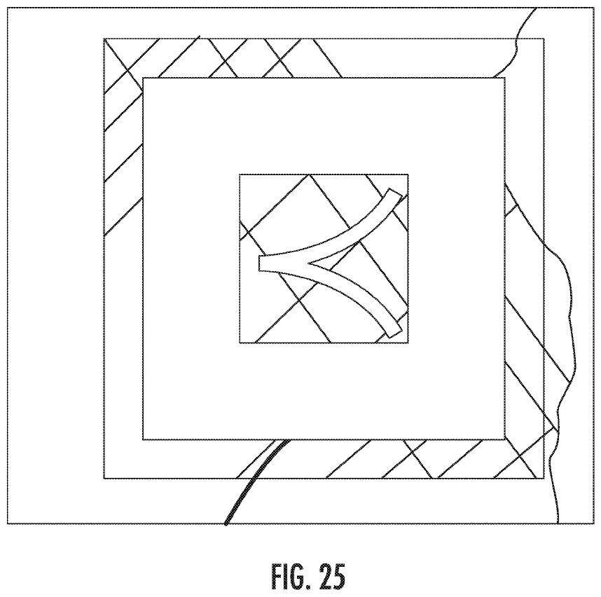

Another method of making the marker is to 3D print a radiotranslucent “well” which can be filled with radiopaque liquid contrast agent, as illustrated in FIGS. 24 and 25. FIG. 24 illustrates a perspective view of a radiovisible AR marker with radiotranslucent “well” filled with liquid contrast agent. FIG. 25 illustrates an image view of a fluoroscopic image with radiographic projection of radiovisible AR marker made from radiotranslucent “well” filled with liquid contrast agent.

Another alternative method could be to print liquid contrast agent onto a paper surface using a modified inkjet printer. In this way variable levels of radiopacity could be laid down at desired locations to form the desired pattern for the AR marker.

The present invention includes a radiovisible augmented reality marker with at least the following fundamental features: the AR marker is recognizable by a camera and virtual information can be mapped to it; the AR marker has a radiographic projection that is visually detectable in fluoroscopic/radiographic images. Additional features include: the orientation of the AR marker in the world frame of reference is linked to its orientation in the image frame of reference; the projectional appearance of the AR marker on the radiograph shows how the x-ray beam impacted it in the physical world frame; the radiographic projection of the AR marker is also recognizable by the camera so that virtual information can be mapped to it (The radiographic projection of the AR marker is itself an AR marker).

It is useful as a fiducial in an image to world registration augmented reality surgical system. Furthermore, it is useful for automatic registration of an image frame of reference to a world frame of reference. Lastly, it can be used to augment a conventional physical monitor with virtual information so as to allow interaction in the image frame of reference that is visible in the world frame of reference or interaction in the world frame of reference that is visible in the image frame of reference.

The implementation of the present invention can be carried out using a computer, non-transitory computer readable medium, or alternately a computing device or non-transitory computer readable medium incorporated into the system, the HMD, or networked in such a way that is known to or conceivable to one of skill in the art.

A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape. The computing device can be a special computer designed specifically for this purpose. The computing device can be unique to the present invention and designed specifically to carry out the method of the present invention. The operating console for the device is a non-generic computer specifically designed by the manufacturer. It is not a standard business or personal computer that can be purchased at a local store. Additionally, the console computer can carry out communications with the surgical team through the execution of proprietary custom built software that is designed and written by the manufacturer for the computer hardware to specifically operate the hardware.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention. While exemplary embodiments are provided herein, these examples are not meant to be considered limiting. The examples are provided merely as a way to illustrate the present invention. Any suitable implementation of the present invention known to or conceivable by one of skill in the art could also be used.

1. A system for image to world registration using a world spatial map comprising:
a spatial mapping apparatus having a visual field for display of information;
a non-transitory computer readable medium programmed for:
receiving image information;
linking positional information in an image frame of reference to corresponding positional information in a world frame of reference and;
displaying a visual representation of the positional information in the image frame of reference linked to the corresponding positional information in the world frame of reference in the visual field displayed by the spatial mapping apparatus.

2. The system of claim 1 further comprising generating visual representations in a 2D image frame of reference that are visualized in a 3D world frame of reference.

3. The system of claim 1 further comprising a radiovisible augmented reality marker.

4. The system of claim 1 further comprising a head mounted display (HMD).

5. The system of claim 4 wherein the HMD uses Simultaneous Localization and Mapping (SLAM).

6. The system of claim 1 further comprising creating a template on the imaging frame of reference in two dimensions and subsequently allowing the user to visualize this template in three dimensions in the world space.

7. The system of claim 1 further comprising scanning the environment as a whole and linking the image information to this reference frame.

8. The system of claim 1 further comprising generating a workflow that mimics a surgeons preferred workflow.

9. The system of claim 1 further comprising displaying a virtual line that is perpendicular to a plane of the image that intersects a point of interest.

10. The system of claim 9 wherein the point of interest lies at any point along the virtual line.

11. The system of claim 9 further comprising displaying the virtual line in a user's field of view in a head mounted display.

12. The system of claim 1 further comprising overlapping world spatial maps and a tracker rigidly fixed to a medical imaging system.

13. A device for labeling in an image comprising:
an augmented reality marker configured to be recognizable by a camera such that virtual information can be mapped to the augmented reality marker; and
a radiographic projection configured to be visually detectable in a fluoroscopic/radiographic image.

14. The device of claim 13 further comprising the augmented reality marker having an orientation in a world frame of reference that is linked to an orientation in an image frame of reference.

15. The device of claim 13 further comprising a projectional appearance of the augmented reality marker showing how an X-Ray beam impacted it in a physical world.

16. The device of claim 13 further comprising the radiographic projection being recognizable by a camera such that virtual information can be mapped to the radiographic projection.

17. The device of claim 16 wherein the virtual information is configured to be displayed via a head mounted display (HMD).

18. The device of claim 13 wherein an orientation of the augmented reality marker in a world frame of reference is linked to its orientation in an image frame of reference.

19. The device of claim 13 wherein an orientation and relation of an X-ray beam to the augmented reality marker is determined from a projectional appearance of the augmented reality marker in a resultant radiographic image.

20. The device of claim 19 further comprising configuring the augmented reality marker to be positioned such that translations are performed in a same plane as a projectional image.
